(19) United States
(12) Patent Application Publication
(10) Pub. No.: US 2009/0185980 A1
(43) Pub. Date: Jul. 23, 2009

(54) IN VIVO DRUG SCREENING SYSTEM

(75) Inventors: CHEN-YUAN DONG, Taipei City (TW); HSUAN-SHU LEE, Taipei City (TW); FENG-CHIEH LI, Taipei City (TW); TZU-LIN SUN, Taipei City (TW); YUAN LIU, Taipei City (TW); PO-SHOU CHANG, Taipei City (TW)

Correspondence Address: WPAT, PC;INTELLECTUAL PROPERTY ATTORNEYS, 2030 MAIN STREET, SUITE 1300, IRVINE, CA, 92614, US

(73) Assignee: NATIONAL TAIWAN UNIVERSITY, Taipei City (TW)

(21) Appl. No.: 12/018,769

(22) Filed: Jan. 23, 2008

(51) Int. Cl. A61K 49/00; A61M 31/00

(52) U.S. Cl. 424/9.2; 604/288.01

(57) ABSTRACT

The present invention provides imaging devices. The present invention also provides in vivo drug screening systems. The present invention further provides a method for in vivo drug screening.

### FIELD OF THE INVENTION

The present invention relates to imaging devices. The present invention also relates to in vivo drug screening systems. The present invention further relates to a method for in vivo drug screening.

### BACKGROUND OF THE INVENTION

The development process of a new drug comprises several time-consuming and costly steps. In the United States, from basic research, laboratory and animal testing, phase I to phase III clinical trials to FDA approval, it takes approximately 15 years and 0.9 billion USD for a new drug to enter the market. Literally hundreds and sometimes thousands of chemical compounds must be made and tested in an effort to find one that can achieve a desirable result. There is no standard route through which drugs are developed. A pharmaceutical company may decide to develop a new drug aimed at a specific disease or medical condition. Sometimes, scientists choose to pursue an interesting or promising line of research. In other cases, new findings from university, government, or other laboratories may point the way for drug companies to follow with their own research.

New drug research starts with an understanding of how the body functions, both normally and abnormally, at its most basic levels. The questions raised by this research help determine a concept of how a drug might be used to prevent, cure, or treat a disease or medical condition. This provides the researcher with a target. Sometimes, scientists find the right compound quickly, but usually hundreds or thousands must be screened. In a series of test tube experiments called assays, compounds are added one at a time to enzymes, cell cultures, or cellular substances grown in a laboratory. The goal is to find which additions show some effect. This process may require testing hundreds of compounds since some may not work, but will indicate ways of changing the compound's chemical structure to improve its performance. Computers can be used to simulate a chemical compound and design chemical structures that might work against it. Enzymes attach to the correct site on a cell's membrane, which causes the disease. A computer can show scientists what the receptor site looks like and how one might tailor a compound to block an enzyme from attaching there. But even though computers give chemists clues as to which compounds to make, a substance must still be tested within a living being. Another approach involves testing compounds made naturally by microscopic organisms. Candidates include fungi, viruses and molds, such as those that led to penicillin and other antibiotics. Scientists grow the microorganisms in what is known as a “fermentation broth,” with one type of organism per broth. Sometimes, 100,000 or more broths are tested to see whether any compound made by a microorganism has a desirable effect.

Before entering to clinical tests, an animal or nonclinical test is required. In animal testing, drug companies make every effort to use as few animals as possible and to ensure their humane and proper care. Generally, two or more species (one rodent, one non-rodent) are tested because a drug may affect one species differently from another. Animal testing is used to measure how much of a drug is absorbed into the blood, how it is broken down chemically in the body, the toxicity of the drug and its breakdown products (metabolites), and how quickly the drug and its metabolites are excreted from the body. Short-term testing in animals ranges in duration from 2 weeks to 3 months, depending on the proposed use of the substance. Long-term testing in animals ranges in duration from a few weeks to several years. Some animal testing continues after human tests begin to learn whether long-term use of a drug may cause cancer or birth defects. Much of this information is submitted to FDA when a sponsor requests to proceed with human clinical trials. The FDA reviews the preclinical research data and then makes a decision as to whether to allow the clinical trials to proceed.

The introduction of Multiphoton fluorescence microscopy revolutionized bioimaging at the submicron level. A number of advantages are associated with this approach. First, the high intensity light source required to induce the non-linear optical effects limits the sample excitation to the focal volume. Bioimaging achieved by scanning the point-like excitation volume results in images with excellent axial depth discrimination. Moreover, the point-like excitation volume also limits the specimen photodamage to the focal volume, thus can prolong the sample longevity. Lastly, the near-infrared photons commonly used to induce multiphoton processes in biological specimens are absorbed and scattered less than the ultraviolet or visible photons used in one-photon microscopy (Peter T. C. So, Chen Y. Dong. Annu. Rev. Biomed. Eng. 2000, 02:399-429).

U.S. Pat. Application Ser. No. 2003/0009104 discloses a method of detecting a neurodegenerative disease in a mammal by activating brain tissue of the mammal by application of radiation under conditions effective to promote a simultaneous multiphoton excitation of the brain tissue and to emit a fluorescence characteristic.

### BRIEF DESCRIPTION OF THE DRAWINGS

FIG. 1 shows the structure of the hepatic imaging chamber and its anatomic position after installation on the mouse abdominal wall. (a) Horizontal and (b) oblique views of the chamber apparatus. (c) A photograph showing a mouse with the device installed on its upper abdominal wall. (d) A diagram depicting the anatomic relationships of the installed device with skin, peritoneum, and the underlying liver.

FIG. 2 shows the structure of the modified hepatic imaging chamber.

FIG. 3 shows accommodation of the mouse on the microscope stage. Fixing of the mouse with its liver chamber into the groove of a steel plate was shown in (a)-(d). The plate is fixed onto the microscope stage by screws.

FIG. 4 shows the light/signal path. The excitation light source comes from Ti-Saphire pulse laser, and then focus on the sample through a series of optics (including a set of mirrors for scanning in X and Y direction). The autofluorescence and second harmonic generation signal from the sample is collected through the objective on the PMT (photo multiplier tube) and then the computer. The scanning image can be obtained from the computer by changing the position of the excitation focusing spot through the scanning mirrors.

FIG. 5 shows a brief schematic of the imaging light path.

FIG. 6 shows the in vivo liver image obtained from the mouse with liver chamber with sinusoids and bile duct.

FIG. 7 shows the image with leukocyte cells crawl on the blood vessel. The light arrow point out where the leukocyte cell specified by Rhodamine 6 G is. The dark arrow pointed out red blood vessel.

FIG. 8 shows fibrosis of liver after feeding CCl₄ for 10 days.

FIG. 9 shows the metabolism of normal hepatocytes over time. When hepatocytes start metabolism, CF increases until reaching maximum at 8-minute and then decreases until the metabolism is completed.

FIG. 10 shows the quantified schema of fluorescence represented the metabolism of normal hepatocytes over time. X-axis indicates the intensity of fluorescence; Y-axis indicates the time of metabolism. This schema corresponds to the image of FIG. 9.

FIG 11 shows the variation of bile metabolism with normal and ligated bile duct over time. BDL indicates the bile metabolism of ligated mouse. X-axis indicates time. Y-axis indicates the intensity of CF (fluorescent dye).

### SUMMARY OF THE INVENTION

The present invention provides imaging devices. The present invention also provides in vivo drug screening systems. The present invention further provides a method for in vivo drug screening.

### DETAILED DESCRIPTION OF THE INVENTION

The present invention is directed to facilitate the development process of a new drug; it provides an in vivo drug screening system, which is used to monitor the effect of the drug in tissues or organs of a tested animal directly and lively. This approach is especially useful for mid- to long-term observation. Through the system, the number of tested animals as well as the cost and time can be reduced while the accuracy and credibility of the test are improved.

The system of the present invention comprises an intravital imaging chamber, which is installed on an affected tissue or organ of the tested animal such as a rodent or a mammal. The tested animal carrying the imaging chamber can live normally. In combination with multiphoton and second harmonic generation microscopy, the live image recording the activities of the targeted tissue or organ with highly spatial resolution is obtained. The processes of installation and observation are very simple using only basic operation and can be completed in 30 minutes or shorter. Several candidate chemical compounds or drugs can be tested at the same time and the tested animals can be reduced to statistic minimum for each drug.

Since liver is the chemical factory in body responsible for important functions such as metabolism and detoxification, the activities of liver are especially worth of observation when evaluating the functions and toxicities of a new drug. In preferred embodiments, the imaging chamber is designed to be a hepatic imaging chamber.

Accordingly, the present invention provides an imaging device comprising:
(I) a lid consisting of
   (i) a body with a hollow through the body;
   (ii) an inner wall of the body;
   (iii) an outer wall of the body, which has one or more groove on the wall;
   (iv) one or more hole through the body;
(II) a U-shaped plate with an indent region, wherein the region has a thickness for embedding the groove of the lid; and
(III) a cover glass adhered to the lid.

The hole is for use in fixation by stitching on a skin of a subject. The device is used for working under oil immersion objective, wherein oil is dropped on the cover glass.

The present also provides an imaging device comprising:
(I) an inner lid consisting of
   (i) a body with a hollow through the body;
   (ii) a hollow stalk, which is linked to the body and a screw on the stalk;
   (iii) an inner wall of the body;
   (iv) an outer wall of the body;
   (v) one or more hole through the body;
(II) an outer lid consisting of
   (i) a body with a hollow through the body;
   (ii) an inner wall of the body with a screw on the wall;
   (iii) an outer wall of the body;
   (iv) one or more hole through the body;
(III) a cover glass adhered to the inner lid.

The device can further comprising a plate for fixation.

The present invention also provides a system for in vivo drug screening comprising: (a) the described imaging device and (b) a multiphoton microscope. The system can further comprise a photon multiplier tube and a computer.

The present invention also provides a method for in vivo drug screening comprising: (a) installation of the described imaging chamber on a targeted tissue or organ such as liver of a tested animal, (b) applying a drug to the tested animal, (c) accommodation of the tested animal onto the microscopic stage of the multiphoton microscope of claim 9, and (d) observation of the targeted tissue or organ by the multiphoton microscope.

The tested animal can be a mammal (such as a dog, a cat or a monkey) or a rodent (such as a mouse).

Step (a) can be accomplished by stitching, and in order to ensure tight contact of the tissue/organ surface with the cover glass of the chamber and to minimize motional artifacts associated with heart beat and respiration, the adhesive such as tissue adhesive Histoacryl can be further applied for adhering the tissue/organ to the imaging chamber.

Step (c) can be accomplished by sliding the chamber into a U-shaped groove of a plate, fixing the chamber without further sliding, putting the plate with the tested animal onto a round ring which can change the direction of the tested animal, and placing the tested animal with the ring on the microscopic stage.

### EXAMPLE

The examples below are non-limiting and are merely representative of various aspects and features of the present invention.

### Example 1

### Design of the Intravital Hepatic Imaging Chamber

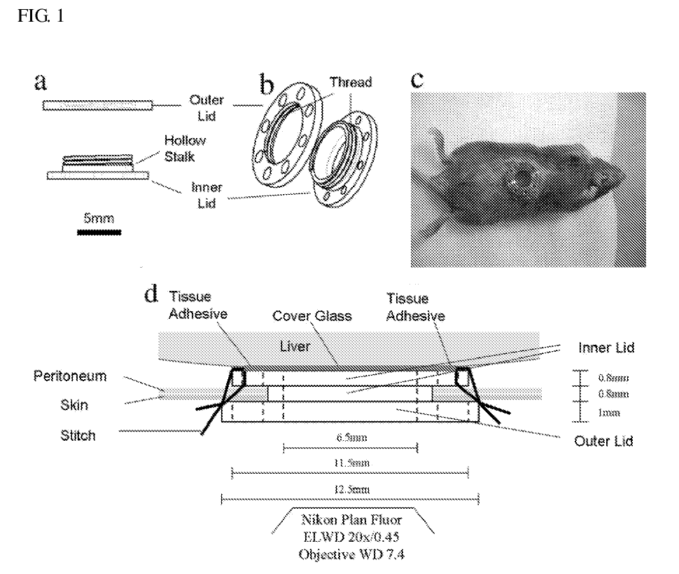

The hepatic imaging chamber was composed of two doughnutshaped titanium rings: the outer and inner lids (FIGS. 1a and 1b). The outer and inner diameters of the outer lid were 12.5 and 6.5 mm, respectively, with a thickness of 1 mm. For the inner lid, the outer and inner diameters were 11.5 and 6.5 mm, respectively, and it had a thickness of 0.8 mm. Both lids were threaded and could be fastened together by simple screwing.

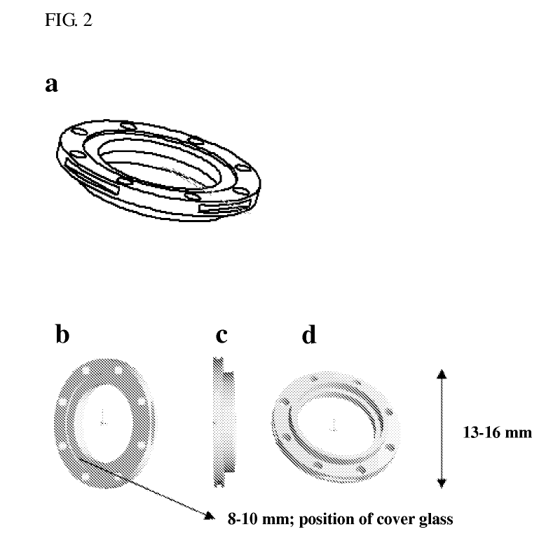

For the in vivo observation, the chamber was further modified by using the oil immersion object and bio-compatible titanium alloy (6V4A1-Ti, ELI grade) to get better spatial resolution and adopted the setup in different in vivo observations without affecting the actual living environment of the organism (FIGS. 2a-d). This modification reduced original three pieces into one. The chamber had doughnut-shaped rings. The outer and inner diameters of the liver chamber were 13-16 and 8-10 mm, respectively, with a thickness of 1 mm.

### Example 2

### Installation of the Hepatic Imaging Chamber on the Mouse

C57BL/6 mice beyond 5 weeks of age were anesthetized using intraperitoneal injection of 2-2-2 tribromoethanol at a dose of 0.35 mg/g. The hairs on the abdomen were shaved and a vertical incision of the skin and peritoneum was made. To visualize the sinusoids, the inferior vena cava was exposed for the injection of 10 mg/mice of rhodamine B isothiocyanate-dextran 7000 (Sigma, Saint Louis, Mo.). The incision of the lower abdomen was sutured, leaving the upper part open to create a circled wound to position the inner lid of the hepatic imaging chamber inside the abdominal wall. Prior to installing the inner lid, an 8-mm round cover glass was adhered around the outer rim by a polyvinylacetate glue. The cover glass serves as the observation window of intravital hepatic activities. The inner lid was then sutured through the small holes on the ring to the skin and peritoneum. To ensure tight contact of the liver surface with the cover glass and to minimize motional artifacts associated with heartbeat and respiration, the adhesive such as tissue adhesive Histoacryl (B. Braun Melsungen AG, Germany) was applied to the edge of the inner lid to adhere the liver to the imaging chamber. For gluing purposes, we tested a number of adhesives. While some such as fibrin did not glue well, others appeared to cause changes in the appearance of the liver surface (not all adhesives were tested under ex-vivo conditions for liver appearance changes). Therefore, the significant criteria of glue selection rest on how well the adhesive can attach the liver to the chamber and the glue's biocompatibility. For comparison purposes, we also acquired images of the hepatic metabolic activities of 6-CFDA using 4011 (for medical device, Henkel Loctite, Rocky Hill, Conn.) and 406 (instant adhesive, Henkel Loctite). In all cases, the tissue adhesive was not applied to the imaged regions. Finally, the outer lid was attached to the inner lid by screwing (FIGS. 1c and 1d).

For installation of the modified hepatic imaging chamber on the mouse, C57BL/6 mice beyond 5 weeks of age were anesthetized using intraperitoneal injection of avertin at a dose of 0.267 mg/g. The hairs on the abdomen were shaved and the circled wound was cut on the skin about 10 mm to position the liver chamber inside the abdominal wall. Then liver chamber and abdomen skin were assembled together with stitches. A 10-mm round cover glass was adhered around the outer rim by polyvinyl acetate glue. The intravital hepatic activities were observed through the cover glass. To ensure tight contact of the liver surface with the cover glass and to minimize motional artifacts associated with heartbeat and respiration, the adhesive such as tissue adhesive Histoacryl B. Braun Melsungen AG, Germany, was applied to surface of cover glass to position the liver close to the imaging chamber.

### Example 3

### Accommodation of the Mouse onto the Microscopic Stage

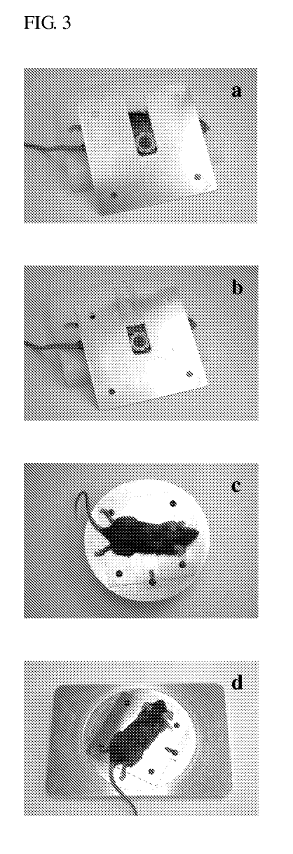

The positioning of the mouse installed with the hepatic imaging chamber and the subsequent imaging procedures are illustrated in FIG. 3. The mouse was kept in the supine position with the hepatic imaging chamber fitted into the U-shaped groove of a steel plate (FIG. 3a), and the liver chamber was slid along gap of the U-shaped plate. A piece of steel was used to steadily fix the chamber without further sliding (FIG. 3b). The mouse with the steel plate was put onto a round ring (FIG. 3c), which could change the direction of the mouse to satisfy the working parameters of the stage. The mouse with the ring was then placed on the microscopic stage of an inverted microscope (TE2000, Nikon, Japan) by screwing the plate onto the microscopic stage (FIG. 3d). In this manner, the mouse can be firmly attached for multiphoton imaging purposes. A hot pack between the gauze was put on top of the mouse for warming purposes.

### Example 4

### Multiphoton Microscopy

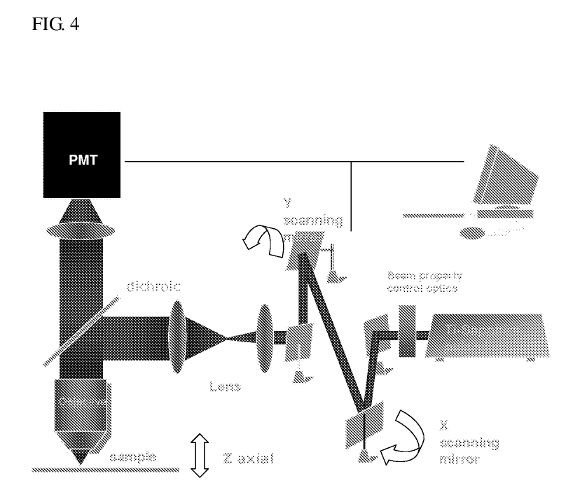

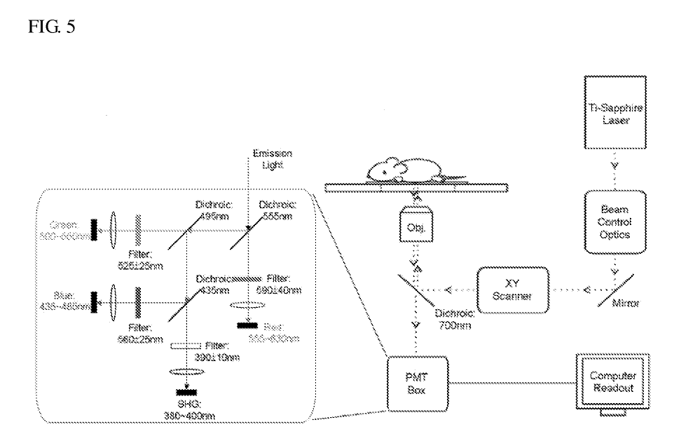

Two-photon microscopy was used to observe living organism. It had the advantages of high penetration depth, low photo-damage, optically slicing of the sample and ability to construct 3D images. The excitation light source came from Ti-Saphire pulse laser, and then focus on the sample through a series of optics (including a set of mirrors for scanning in X and Y direction). The autofluorescence and second harmonic generation signal from the sample was collected through the objective on the PMT (photon multiplier tube) and then the computer, and by changing the position of the excitation focusing spot through the scanning mirrors, the scanning image was obtained from the computer (FIG. 4). The multiphoton microscope is illustrated in FIG. 5. A titanium-sapphire laser with 780-nm output (Tsunami, Spectra Physics, Mountain View, Calif.) pumped by a diodepumped solid-state laser (Millennia X, Spectra Physics) was used for excitation. The laser was scanned by an x-y mirror scanning system (Model 6220, Cambridge Technology, Cambridge, Mass.) and guided toward the modified inverted microscope. The laser was beam expanded and reflected into the back aperture of a long working distance (7.4 mm) objective (Plan Fluor ELWD 20), NA 0.45, Nikon) by a primary dichroic mirror (700DCSPXRUV-3p, Chroma Technology, Rockingham, Vt.). The power at the sample was around 21 mW, and sample luminescence was collected in the epi-illuminated or backscattering geometry. After passing through the primary dichroic mirror, the second harmonic generation (SHG) and fluorescence signals were separated into four simultaneous detection channels by secondary dichroic mirrors (435DCXR, 495DCXR, 555DCLP, Chroma Technology) and additional bandpass filters (HQ390/20, HQ460/50, HQ525/50, HQ590/80, Chroma Technology). The detection bandwidths for the SHG, blue, green, and red fluorescence were 390±10, 460±25, 525±25, and 590±40 nm, respectively. The SHG signal was collected in the backscattering geometry. Single-photon counting photomultiplier tubes (R7400P, Hamamatsu, Japan) were used as optical detectors. Each optical scan was composed of 256×256 pixels and took approximately 4 s to complete. For image processing, the software of ImageJ (National Institute of Health, Bethesda, Md.) and Meta-Morph (Universal Imaging Corporation, Downingtown, Pa.) were used. Right before the visualization of hepatobiliary excretory dynamics, we would inject 50 μg/mouse of 6-carboxyfluorescein diacetate (6-CFDA, Sigma) either through the tail or jugular veins, and began to acquire the serial multiphoton images at 4-s intervals. The procedures of the animal experiments were approved by the Institutional Laboratory Animal Care Committee of National Taiwan University, College of Medicine.

### Example 5

### Multiphoton Images Acquired using Histoacryl

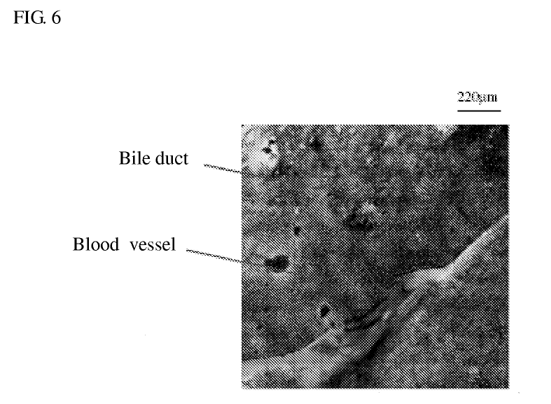

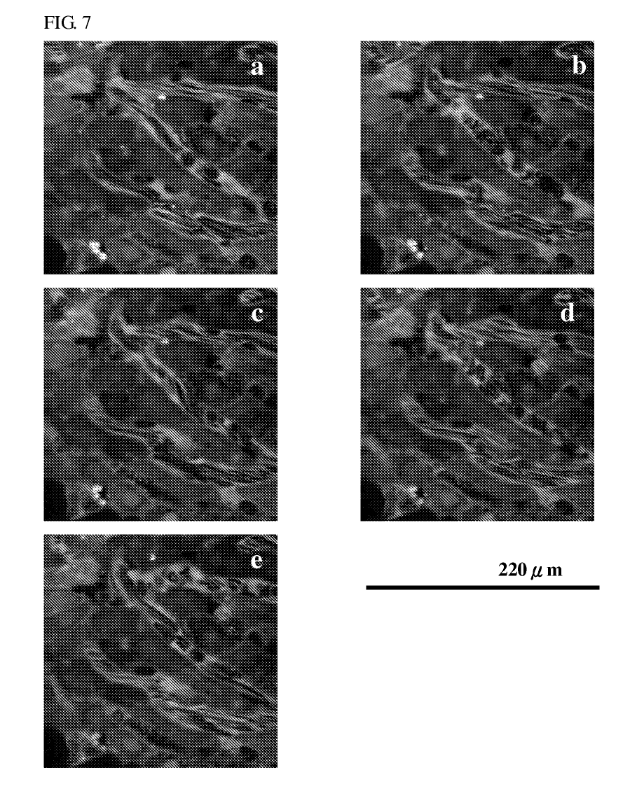

The depth for imaging hepatobiliary function was indicated by the disappearance of the SHG signals from the capsule. Upon injection of 6-CFDA through the jugular vein, the sinusoids were already demarcated by the red fluorescence injected from tail vein. To visualize the sinusoids, the inferior vena cava was injected with 10 mg/mice of rhodamine Bisothiocyanate-dextran 70000 (Sigma, Saint Louis, Mo.). FIG. 6 shows the representative multiphton images of mouse installed with liver chamber. Images in FIG. 6 were obtained through 20X oil immersion objective and shown partial liver of the mouse, in which the sinusoids demarcated by rhodamine obviously were distributed in the liver and bile ducts demarcated by CFDA. The large image was flat and steady without any disturbance. At this moment, mouse was still alive and observations could be carried on. FIG. 7 shows image of leukocytes crawled on the wall of blood inside the sinusoids. Leukocytes were demarcated by Rhodamine 6G.

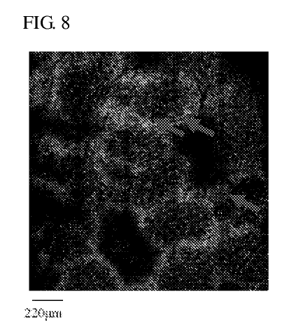

When the liver was damaged, the function of hepatocytes would also be damaged. In the present invention, CCl₄ was applied to simulate the reaction of hepatocyte when the liver was toxic. After feeding CCl₄ for 10 days, the liver was fibrosis (FIG. 8). This approach can be used to screen drugs which can repair hepatocytes.

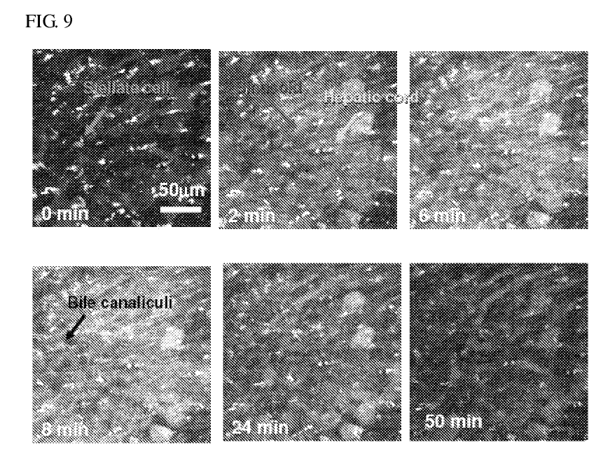

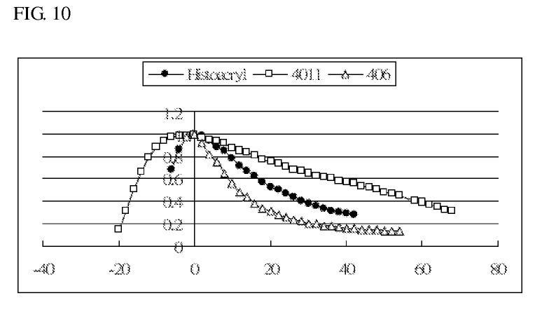

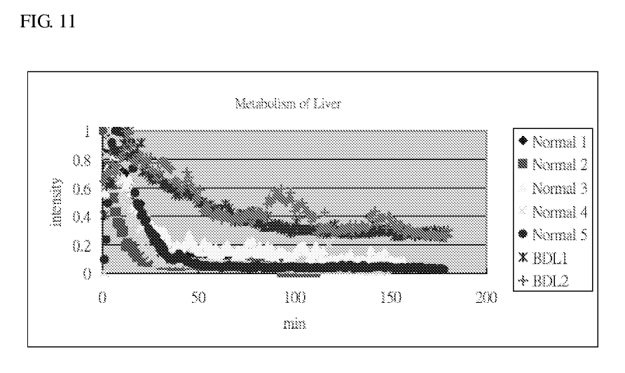

The bile metabolism process of normal hepatocytes was recorded by the system (FIGS. 9, 10). We also ligated the bile duct to simulate cholestasis. The damage of hepatocytes was cause by abnormal metabolism of bile. This approach can be used to screen drugs which facilitate bile metabolism (FIG. 11).

While the invention has been described and exemplified in sufficient detail for those skilled in this art to make and use it, various alternatives, modifications, and improvements should be apparent without departing from the spirit and scope of the invention.

What is claimed is:

1. An imaging device comprising:
(I) a lid consisting of
(i) a body with a hollow through the body;
(ii) an inner wall of the body;
(iii) an outer wall of the body, which has one or more groove on the wall;
(iv) one or more hole through the body;
(II) a U-shaped plate with an indent region, wherein the region has a thickness for embedding the groove of the lid; and
(III) a cover glass adhered to the lid.

2. The device of claim 1, wherein the hole is for use in fixation by stitching on a skin of a subject.

3. The device of claim 1, which is for working under oil immersion objective.

4. An imaging device comprising:
(I) an inner lid consisting of
(i) a body with a hollow through the body;
(ii) a hollow stalk, which is linked to the body and a screw on the stalk;
(iii) an inner wall of the body;
(iv) an outer wall of the body;
(v) one or more hole through the body;
(II) an outer lid consisting of
(i) a body with a hollow through the body;
(ii) an inner wall of the body with a screw on the wall;
(iii) an outer wall of the body;
(iv) one or more hole through the body;
(III) a cover glass adhered to the inner lid.

5. The device of claim 4, which further comprising a plate for fixation.

6. A system for in vivo drug screening comprising: (a) the imaging chamber of claim 1 and (b) a multiphoton microscope.

7. The system of claim 6, wherein the excitation light source of the multiphoton microscope comes from titanium-sapphire pulse laser with 780 nm output, and the laser was scanned by an x-y mirror scanning system.

8. The system of claim 6, which further comprises a photon multiplier tube and a computer.

9. A system for in vivo drug screening comprising: (a) the imaging chamber of claim 4 and (b) a multiphoton microscope.

10. The system of claim 9, wherein the excitation light source of the multiphoton microscope comes from titanium-sapphire pulse laser with 780 nm output, and the laser was scanned by an x-y mirror scanning system.

11. The system of claim 9, which further comprises a photon multiplier tube and a computer.

12. A method for in vivo drug screening comprising: (a) installation of the imaging chamber of claim 1 on a targeted tissue or organ of a tested animal, (b) applying a drug to the tested animal, (c) accommodation of the tested animal onto the microscopic stage of the multiphoton microscope of claim 9, and (d) observation of the targeted tissue or organ by the multiphoton microscope.

13. The method of claim 12, wherein the targeted tissue or organ is liver.

14. The method of claim 12, wherein the tested animal is a mammal.

15. The method of claim 12, wherein the tested animal is a rodent.

16. The method of claim 12, wherein step (a) is accomplished by stitching.

17. The method of claim 16, wherein step (a) is further accomplished by applying adhesive for adhering the targeted tissue or organ to the chamber.

18. The method of claim 12, wherein step (c) is accomplished by sliding the chamber into a U-shaped groove of a plate, fixing the chamber without further sliding, putting the plate with the tested animal onto a round ring which can change the direction of the tested animal, and placing the tested animal with the ring on the microscopic stage.
